(19) United States
(12) Plant Patent Application Publication
(10) Pub. No.: US 2007/0300340 P1
(43) Pub. Date: Dec. 27, 2007

(54) HEUCHERA PLANT NAMED 'CINNABAR SILVER'

(75) Inventor: Janet N. Egger, Wilsonville, OR (US)

Correspondence Address: KLARQUIST SPARKMAN, LLP, 121 SW SALMON STREET, SUITE 1600, PORTLAND, OR, 97204, US

(73) Assignee: Terra Nova Nurseries, Inc.

(21) Appl. No.: 11/474,026

(22) Filed: Jun. 23, 2006

(51) Int. Cl. A01H 5/00

(52) U.S. Cl. PLT/263

(57) ABSTRACT

A new and distinct hybrid of Heuchera plant characterized by medium sized, dark leaves with heavy silver veiling; brick red colored flowers; compact, mounding habit; excellent vigor.

### BOTANICAL DENOMINATION

Heuchera hybrid

### VARIETY DESIGNATION

‘Cinnabar silver’

The present invention relates to a new and distinct cultivar of Heuchera hybrid and given the cultivar name of ‘Cinnabar Silver’. Heuchera is in the family Saxifragaceae. Heuchera ‘Cinnabar Silver’ originated from a cross between Heuchera 45-1 (an unpatented, proprietary plant) as the seed parent, and Heuchera 68-2 (an unpatented, proprietary plant) as the pollen parent. Given its ornamental foliage and flower, it is probable that Heuchera sanguinea and H. americana are in its parentage.

This new Heuchera is distinguished by:
1. Medium sized, dark leaves with heavy silver veiling.
2. Brick red colored flowers.
3. Compact, mounding habit.
4. Excellent vigor.

This new cultivar has been reproduced only by asexual propagation (division and tissue culture). Each of the progeny exhibits identical characteristics to the original plant. Asexual propagation by division and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

FIG. 1 shows a three-year-old plant of Heuchera ‘Cinnabar Silver’ growing in a two gallon container in the shade house in April in Canby, Oreg. FIG. 2 shows the same plant the year before grown under higher light conditions.

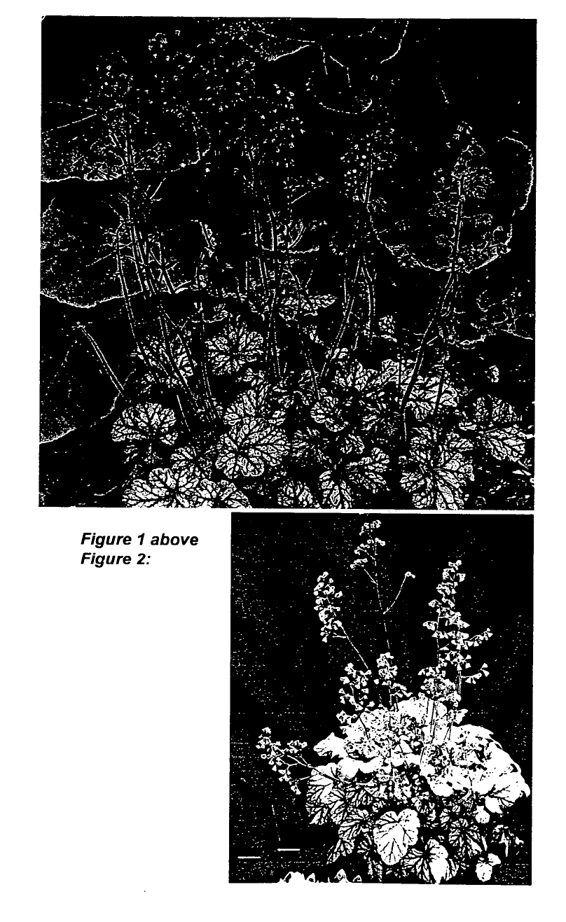

### DETAILED PLANT DESCRIPTION

The following is a detailed description of the new Heuchera cultivar based on observations taken in May of a three-year-old specimen grown a three gallon container in an open air shade house with 50% shade cover and added irrigation in the summer months in Canby, Oreg. Canby is Zone 8 on the USDA Hardiness map. Temperatures range from a high of 95 degrees F. in August to 32 degrees F. in January. Normal rainfall in Canby is 42.8 inches per year. The color descriptions are all based on The Royal Horticultural Society Colour Chart.
Botanical classification: Heuchera hybrid.
Variety denomination: ‘Cinnabar Silver’.
Parentage: Heuchera 45-1 (seed parent)×Heuchera 68-1 (pollen parent).
Plant:
   Form.—Basal rosette, herbaceous perennial.
   Hardiness.—USDA Zone 4 to 9.
   Size.—22 cm tall from the ground to the top of the foliage and 40 cm wide.
   Habit.—Mounding.
   Vigor.—Excellent.
   Roots.—Fibrous, freely branching, fine, and white in color. Roots develop easily from cuttings.
Leaf:
   Type.—Simple.
   Arrangement.—Rosette.
   Shape.—Broadly ovate, flattened appearance.
   Lobing/division.—5 to 7 shallow lobes.
   Venation.—Palmate.
   Margins.—Crenate.
   Apex.—Obtuse.
   Base.—Cordate, lobes sometimes slightly overlapping at the base.
   Blade size.—8.5 to 10 cm long and 8.5 to 9 cm wide.
   Surface texture.—Sparsely glandular on both sides, satiny in appearance, leathery feeling.
   Petiole size.—10 cm long and 2 to 3 mm wide.
   Petiole surface texture.—Glandular — pubescent.
   Petiole color.—Brown 200D.
   Leaf color.—Spring, summer topside — Before flowering the new leaves are deep purple, Greyed Purple 187A with a purple silver veil, Violet 87C. Once the flowers open the leaf color is silver, Grey Green 198D with veins Grayed Purple 187A Bottom side all seasons — Between Grayed Purple 187A and Red Purple 59A and shiny Winter topside — Silver, Greyed Green 198D with veins between Yellow Green 147A and Brown 200A.
Inflorescence:
   Type.—Thyrse.
   Number of flowers.—150 per thyrse.
   Number of thyrse in spring.—30 in first flush.
   Peduncle.—With 0 to 3 petiolate cauline leaves, ovate, blades grow to 3 cm long and 3 cm wide, margin crenate, base usually cordate. Height — 46 cm Diameter — 0.5 cm near base Color — Greyed Purple 187A Surface texture — Glandular hairs.
   Pedicel.—Length — Variable. Surface texture-Glandular hairs Color-Grayed Purple 187A.
   Bloom period.—May in Canby, Oreg.
Flower bud:
   Size.—2 mm wide and 4 mm long.
   Description.—Glandular puberulent, ovoid.
   Color.—Pink, Greyed Purple 185D at base and Greyed Yellow 160C at tip.
Flower:
   Type.—Perfect, zygomorphic.
   Shape.—Campanulate.
   Size.—7 mm long and 7 mm wide.
   Petal description.—5 in number, reflexed, 3 mm long and 0.8 mm wide, glandular on backs, margins mostly entire, tip acute, base attenuate, bright pink, Red 52B.
   Calyx description.—7 mm deep and 7 mm wide, 5 lobes, ovate, cut ¼ way to the base, with glandular hairs, reflex, tip acute, margins entire, inside color is between Greyed Red 179A and Red 47A, outside base is Greyed Red 178A, middle and lobes are Greyed Red 179A.
   Stamen description.—5, reduced, filaments White 155A, 2 mm long, anthers sterile, Greyed Yellow 161C.
   Pistil.—6 mm long, Yellow Green 154C, two beaked.
   Fragrance.—None.
   Lastingness.—Each thyrse blooms for about 2 weeks on the plant.
Fruit:
   Type.—Two-beaked ovoid capsule.
   Color.—Brown 200D.
Seed:
   Shape.—Linear.
   Size.—2 mm. long.
   Color.—Black. RHS 202A.
Pest and disease tolerance: Excellent disease resistance to powdery mildew, the common problem of Heuchera. Susceptible to Heuchera rust and root weevils.

### COMPARISON TO SIMILAR HEUCHERAS

Compared to Heuchera ‘Swirling Fantasy’ (U.S. Plant Pat. No. 14,542), Heuchera ‘Cinnabar Silver’ has brick red colored flowers rather than pinkish red. The new cultivar has shorter flower stalks and much more leaf silvering.

Compared to Heuchera ‘Cherries Jubilee’ (U.S. Plant Pat. No. 11,377), the new variety has larger, darker flowers and darker, heavily veiled leaves.

Compared to the seed parent, Heuchera 45-1 (an unpatented, proprietary plant), the new variety has shorter flowering stems and larger flowers of brick red rather than dull red.

Compared to the pollen parent, Heuchera 68-2 (an unpatented, proprietary plant), the new variety has darker leaves, a heavier veil, and brick red rather than dull orange red flowers.

I claim:

1. A new and distinct hybrid of Heuchera plant as herein illustrated and described.
